(19) United States
(12) Patent Application Publication
(10) Pub. No.: US 2010/0204275 A1
(43) Pub. Date: Aug. 12, 2010

(54) N-PIPERIDIN-4-YLMETHYL-AMIDE DERIVATIVES AND THEIR USE AS MONOAMINE NEUROTRANSMITTER RE-UPTAKE INHIBITORS

(75) Inventors: Dan Peters, Malmo (SE); Birgitte L. Eriksen, Farum (DK); John Paul Redrobe, Rodovre (DK); Gordon Munro, Kobenhavn (DK); Elsebet Østergaard Nielsen, Kobenhavn (DK)

Correspondence Address: BIRCH STEWART KOLASCH & BIRCH, PO BOX 747, FALLS CHURCH, VA, 22040-0747, US

(73) Assignee: NeuroSearch A/S, Ballerup (DK)

(21) Appl. No.: 12/671,317

(22) PCT Filed: Jul. 31, 2008
(86) PCT No.: PCT/EP08/60033

§ 371 Date: Jan. 29, 2010

Related U.S. Application Data
(60) Provisional application No. 60/953,753, filed on Aug. 3, 2007.

(30) Foreign Application Priority Data
Aug. 2, 2007 (DK) PA 2007 01113

(51) Int. Cl. A61K 31/445; C07D 211/26; C07D 401/12; A61K 31/4545; C07D 211/34; A61K 31/4465; A61P 25/00; A61P 25/24; A61P 25/22; A61P 25/28; A61P 25/16; A61P 25/30; A61P 29/00; A61P 13/00

(52) U.S. Cl. 514/318; 546/245; 514/330; 546/193; 514/319; 546/247; 546/246; 514/331

(57) ABSTRACT

This invention relates to novel N-piperidin-4-ylmethyl amide derivatives useful as monoamine neurotransmitter re-uptake inhibitors.

In other aspects the invention relates to the use of these compounds in a method for therapy, and to pharmaceutical compositions comprising the compounds of the invention.

### TECHNICAL FIELD

This invention relates to novel N-piperidin-4-ylmethyl amide derivatives useful as monoamine neurotransmitter re-uptake inhibitors.

In other aspects the invention relates to the use of these compounds in a method for therapy, and to pharmaceutical compositions comprising the compounds of the invention.

### BACKGROUND ART

Serotonin Selective Reuptake Inhibitors (SSRIs) currently provide efficacy in the treatment of several CNS disorders, including depression and panic disorder. SSRIs are generally perceived by psychiatrists and primary care physicians as effective, well-tolerated and easily administered. However, they are associated with a number of undesirable features.

Thus, there is still a strong need for compounds with an optimised pharmaco-logical profile as regards the activity on reuptake of the monoamine neurotransmitters serotonin, dopamine and noradrenaline, such as the ratio of the serotonin reuptake versus the noradrenaline and dopamine reuptake activity.

U.S. Pat. No. 5,885,999 describes the synthesis of the intermediate N-phenyl-N-piperidin-4-ylmethyl-propionamide (Intermediate 8). However, no pharmaceutical use of the compound is disclosed.

WO 2007/093603 describes—in Example 10—the synthesis of eight intermediates, all N-piperidin-4-ylmethyl-amide derivates. No pharmaceutical use of these compounds is disclosed.

### SUMMARY OF THE INVENTION

It is an object of the invention to provide novel compounds which show activity as monoamine neurotransmitter re-uptake inhibitors.

In another aspect, the invention provides a compound of Formula I,

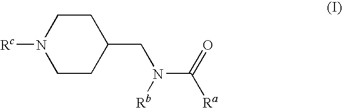

any of its stereoisomers or any mixture of its stereoisomers, or a pharmaceutically acceptable salt thereof; wherein R^{a}, R^{b} and R^{c} are as defined below.

In another aspect, the invention provides a pharmaceutical composition, comprising a therapeutically effective amount of a compound of Formula I, any of its stereoisomers or any mixture of its stereoisomers, or a pharmaceutically acceptable salt thereof, together with at least one pharmaceutically acceptable carrier, excipient or diluent.

In another aspect, the invention provides the use of a compound of Formula I, any of its stereoisomers or any mixture of its stereoisomers, or a pharmaceutically acceptable salt thereof, for the manufacture of a pharmaceutical composition for the treatment, prevention or alleviation of a disease or a disorder or a condition of a mammal, including a human, which disease, disorder or condition is responsive to inhibition of monoamine neurotransmitter re-uptake in the central nervous system.

In another aspect, the invention relates to a method for treatment, prevention or alleviation of a disease or a disorder or a condition of a living animal body, including a human, which disorder, disease or condition is responsive to responsive to inhibition of monoamine neurotransmitter re-uptake in the central nervous system, which method comprises the step of administering to such a living animal body in need thereof a therapeutically effective amount of a compound of Formula I, any of its stereoisomers or any mixture of its stereoisomers, or a pharmaceutically acceptable salt thereof.

Other objects of the invention will be apparent to the person skilled in the art from the following detailed description and examples.

### DETAILED DISCLOSURE OF THE INVENTION

In one aspect, the invention provides a compound of Formula I,

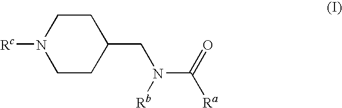

any of its stereoisomers or any mixture of its stereoisomers, or a pharmaceutically acceptable salt thereof; wherein
R^{a} represents hydrogen or alkyl; which alkyl is optionally substituted with one or more substituents independently selected from the group consisting of halo, trifluoromethyl, trifluoromethoxy, cyano, hydroxy, amino, nitro, alkoxy, cycloalkoxy, alkyl, cycloalkyl, cycloalkylalkyl, alkenyl and alkynyl;
R^{b} represents an aryl or heteroaryl group; which aryl or heteroaryl group is optionally substituted with one or more substituents independently selected from the group consisting of halo, trifluoromethyl, trifluoromethoxy, cyano, nitro, hydroxy, alkoxy, cycloalkoxy, phenyloxy, benzyloxy, alkoxyalkyl, cycloalkoxyalkyl, methylenedioxy, ethylenedioxy, alkyl, cycloalkyl, cycloalkylalkyl, alkenyl, alkynyl, sulfanyl, thioalkoxy, —NR′R″, —(C═O)NR′R″ or —NR′(C═O)R″; wherein R′ and R″ independent of each other are hydrogen or alkyl;
R^{c} represents hydrogen or alkyl;
with the proviso that the compound is not
N-(4-Chloro-phenyl)-N-piperidin-4-ylmethyl-propionamide;
N-Benzyl-N-piperidin-4-ylmethyl-propionamide;
N-Phenyl-N-piperidin-4-ylmethyl-propionamide;
N-(3-Methoxy-phenyl)-N-piperidin-4-ylmethyl-propionamide;
N-Benzo[1,3]dioxol-5-yl-N-piperidin-4-ylmethyl-propionamide;
N-(4-Methoxy-phenyl)-N-piperidin-4-ylmethyl-propionamide;
N-(4-Fluoro-phenyl)-N-piperidin-4-ylmethyl-propionamide, or
N-Phenyl-N-piperidin-4-ylmethyl-acetamide.

In one embodiment of the invention, R^{a} represents alkyl. In another embodiment, R^{a} represents methyl or ethyl. In another embodiment, R^{a} represents alkyl substituted with alkoxy.

In another embodiment of the invention, R^{b} represents an optionally substituted aryl group, such as optionally substituted phenyl. In another embodiment, R^{b} represents phenyl. In another embodiment, R^{b} represents halophenyl, such as fluorophenyl, chlorophenyl, 4-halophenyl or 3-halophenyl, e.g. 4-chlorophenyl, 4-fluorophenyl or 3-fluorophenyl. In another embodiment, R^{b} represents dihalophenyl, such as dichlorophenyl or chlorofluorophenyl, such as 3,4-dichlorophenyl, 4-chloro-3-fluorophenyl or 3-chloro-4-fluorophenyl. In another embodiment, R^{b} represents alkoxy-phenyl, such as 4-alkoxyphenyl, 3-alkoxyphenyl or methoxyphenyl, e.g. 4-methoxy-phenyl or 3-methoxyphenyl. In another embodiment, R^{b} represents phenyl substituted with methylenedioxy, such as benzo[1,3]dioxol-5-yl.

In another embodiment of the invention, R^{b} represents optionally substituted naphthyl. In another embodiment, R^{b} represents hydroxynaphthyl, such as hydroxyl-naphthalen-2-yl, such as 6-hydroxpaphthalen-2-yl. In another embodiment, R^{b} represents alkoxynaphthyl, such as methoxynaphthyl, such as methoxynaphthalen-2-yl, such as 6-methoxynaphthalen-2-yl. In another embodiment, R^{b} represents benzyl-oxynaphthyl, such as benzyloxpaphthalen-2-yl, such as 6-benzyloxynaphthalen-2-yl.

In another embodiment of the invention, R^{b} represents optionally substituted pyridyl. In another embodiment, R^{b} represents pyridyl. In another embodiment, R^{b} represents halopyridyl, chloropyridyl, such as chloro-pyridin-2-yl, such as 5-chloro-pyridin-2-yl.

In another embodiment of the invention, R^{e} represents hydrogen. In another embodiment, R^{e} represents alkyl, such as methyl.

In another embodiment the compound of the invention is:
N-(3-Chloro-phenyl)-N-piperidin-4-ylmethyl-propionamide;
N-Phenyl-N-piperidin-4-ylmethyl-propionamide;
N-Piperidin-4-ylmethyl-N-pyridin-2-yl-propionamide;
N-(3,4-Dichloro-phenyl)-N-piperidin-4-ylmethyl-propionamide,
N-(6-Methoxy-naphthalen-2-yl)-N-piperidin-4-ylmethyl-propionamide;
N-(5-Chloro-pyridin-2-yl)-N-piperidin-4-ylmethyl-propionamide;
N-(6-Benzyloxy-naphthalen-2-yl)-N-piperidin-4-ylmethyl-propionamide;
N-(6-Hydroxy-naphthalen-2-yl)-N-piperidin-4-ylmethyl-propionamide; or
   a pharmaceutically acceptable salt thereof.

In another embodiment the compound of the invention is:
N-(3,4-Dichloro-phenyl)-N-piperidin-4-ylmethyl-acetamide;
N-(3,4-Dichloro-phenyl)-2-methoxy-N-piperidin-4-ylmethyl-acetamide;
N-(3,4-Dichloro-phenyl)-N-(1-methyl-piperidin-4-ylmethyl)-propionamide; or
   a pharmaceutically acceptable salt thereof.

In another embodiment the compound for use according to the invention is:
N-(4-Chloro-phenyl)-N-piperidin-4-ylmethyl-propionamide;
N-Benzyl-N-piperidin-4-ylmethyl-propionamide;
N-Phenyl-N-piperidin-4-ylmethyl-propionamide;
N-(3-Methoxy-phenyl)-N-piperidin-4-ylmethyl-propionamide;
N-Benzo[1,3]dioxol-5-yl-N-piperidin-4-ylmethyl-propionamide;
N-(4-Methoxy-phenyl)-N-piperidin-4-ylmethyl-propionamide;
N-(4-Fluoro-phenyl)-N-piperidin-4-ylmethyl-propionamide;
N-Phenyl-N-piperidin-4-ylmethyl-acetamide; or
   a pharmaceutically acceptable salt thereof.

Any combination of two or more of the embodiments described herein is considered within the scope of the present invention.

### Definition of Substituents

In the context of this invention halo represents fluoro, chloro, bromo or iodo.

In the context of this invention an alkyl group designates a univalent saturated, straight or branched hydrocarbon chain. The hydrocarbon chain may contain of from one to six carbon atoms (C₁₋₆-alkyl), including pentyl, isopentyl, neopentyl, tertiary pentyl, hexyl and isohexyl. In another embodiment alkyl represents a C₁₋₄-alkyl group, including butyl, isobutyl, secondary butyl, and tertiary butyl. In another embodiment of this invention alkyl represents a C₁₋₃-alkyl group, which may be methyl, ethyl, propyl or isopropyl.

In the context of this invention an alkenyl group designates a carbon chain containing one or more double bonds, including di-enes, tri-enes and poly-enes. In another embodiment the alkenyl group of the invention comprises of from two to six carbon atoms (C₂₋₆-alkenyl), including at least one double bond. In another embodiment the alkenyl group of the invention is ethenyl; 1- or 2-propenyl; 1-, 2- or 3-butenyl, or 1,3-butadienyl; 1-, 2-, 3-, 4- or 5-hexenyl, or 1,3-hexadienyl, or 1,3,5-hexatrienyl.

In the context of this invention an alkynyl group designates a carbon chain containing one or more triple bonds, including di-ynes, tri-ynes and poly-ynes. In a preferred embodiment the alkynyl group of the invention comprises of from two to six carbon atoms (C₂₋₅-alkynyl), including at least one triple bond. In another embodiment the alkynyl group of the invention is ethynyl; 1-, or 2-propynyl; 1-, 2-, or 3-butynyl, or 1,3-butadiynyl; 1-, 2-, 3-, 4-pentynyl, or 1,3-pentadiynyl; 1-, 2-, 3-, 4-, or 5-hexynyl, or 1,3-hexadiynyl or 1,3,5-hexatriynyl.

In the context of this invention a cycloalkyl group designates a cyclic alkyl group, e.g. containing of from three to seven carbon atoms (C₃₋₇-cycloalkyl), including cyclopropyl, cyclobutyl, cyclopentyl, cyclohexyl and cycloheptyl.

Alkoxy is —O-alkyl, wherein alkyl is as defined above.

Cycloalkoxy means —O-cycloalkyl, wherein cycloalkyl is as defined above.

Cycloalkylalkyl means cycloalkyl as above and alkyl as above, meaning for example, cyclopropylmethyl.

Thioalkoxy is —S-alkyl, wherein alkyl is as defined above.

Amino is NH₂ or NH-alkyl or N-(alkyl)₂, wherein alkyl is as defined above.

In the context of this invention an aryl group designates a carbocyclic aromatic ring system such as phenyl, naphthyl (1-naphthyl or 2-naphthyl) or fluorenyl.

Examples of monocyclic heteroaryl groups of the invention include aromatic 5- and 6-membered heterocyclic monocyclic groups, including for example, but not limited to, oxazolyl, isoxazolyl, thiazolyl, isothiazolyl, tetrazolyl, 1,2,4-oxadiazolyl, 1,2,5-oxadiazolyl, 1,3,4-oxadiazolyl, triazolyl, 1,2,4-thiadiazolyl, 1,2,5-thiadiazolyl, imidazolyl, pyrrolyl, pyrazolyl, furanyl, thienyl, pyridyl, pyrimidyl, or pyridazinyl.

Examples of bicyclic heteroaryl groups of the invention include for example, but not limited to, indolizinyl, indolyl, isoindolyl, indazolyl, benzofuranyl, benzo[b]thienyl, benzimidazolyl, benzoxazolyl, benzooxadiazolyl, benzothiazolyl, benzo[d]isothiazolyl, purinyl, quinolinyl, isoquinolinyl, cinnolinyl, phthalazinyl, quinazolinyl, quinoxalinyl, 1,8-naphthyridinyl, pteridinyl, and indenyl.

### Pharmaceutically Acceptable Salts

The chemical compound of the invention may be provided in any form suitable for the intended administration. Suitable forms include pharmaceutically (i.e. physiologically) acceptable salts, and pre- or prodrug forms of the chemical compound of the invention.

Examples of pharmaceutically acceptable addition salts include, without limitation, the non-toxic inorganic and organic acid addition salts such as the hydro-chloride, the hydrobromide, the nitrate, the perchlorate, the phosphate, the sulphate, the formate, the acetate, the aconate, the ascorbate, the benzenesulphonate, the benzoate, the cinnamate, the citrate, the embonate, the enantate, the fumarate, the glutamate, the glycolate, the lactate, the maleate, the malonate, the mandelate, the methanesulphonate, the naphthalene-2-sulphonate, the phthalate, the salicylate, the sorbate, the stearate, the succinate, the tartrate, the toluene-p-sulphonate, and the like. Such salts may be formed by procedures well known and described in the art.

Examples of pharmaceutically acceptable cationic salts of a chemical compound of the invention include, without limitation, the sodium, the potassium, the calcium, the magnesium, the zinc, the aluminium, the lithium, the choline, the lysinium, and the ammonium salt, and the like, of a chemical compound of the invention containing an anionic group. Such cationic salts may be formed by procedures well known and described in the art.

In the context of this invention the “onium salts” of N-containing compounds are also contemplated as pharmaceutically acceptable salts. Preferred “onium salts” include the alkyl-onium salts, the cycloalkyl-onium salts, and the cycloalkylalkyl-onium salts.

Examples of pre- or prodrug forms of the chemical compound of the invention include examples of suitable prodrugs of the substances according to the invention include compounds modified at one or more reactive or derivatizable groups of the parent compound. Of particular interest are compounds modified at a carboxyl group, a hydroxyl group, or an amino group. Examples of suitable derivatives are esters or amides.

The chemical compound of the invention may be provided in dissoluble or indissoluble forms together with a pharmaceutically acceptable solvent such as water, ethanol, and the like. Dissoluble forms may also include hydrated forms such as the monohydrate, the dihydrate, the hemihydrate, the trihydrate, the tetrahydrate, and the like. In general, the dissoluble forms are considered equivalent to indissoluble forms for the purposes of this invention.

### Steric Isomers

It will be appreciated by those skilled in the art that the compounds of the present invention may exist in different stereoisomeric forms—including enantiomers, diastereomers or cis-trans-isomers.

The invention includes all such stereoisomers and any mixtures thereof including racemic mixtures.

Racemic forms can be resolved into the optical antipodes by known methods and techniques. One way of separating the enantiomeric compounds (including enantiomeric intermediates) is—in the case the compound being a chiral acid—by use of an optically active amine, and liberating the diastereomeric, resolved salt by treatment with an acid. Another method for resolving racemates into the optical antipodes is based upon chromatography on an optical active matrix. Racemic compounds of the present invention can thus be resolved into their optical antipodes, e.g., by fractional crystallisation of D- or L- (tartrates, mandelates, or camphor-sulphonate) salts for example.

The chemical compounds of the present invention may also be resolved by the formation of diastereomeric amides by reaction of the chemical compounds of the present invention with an optically active activated carboxylic acid such as that derived from (+) or (−) phenylalanine, (+) or (−) phenylglycine, (+) or (−) camphanic acid or by the formation of diastereomeric carbamates by reaction of the chemical compound of the present invention with an optically active chloroformate or the like.

Additional methods for the resolving the optical isomers are known in the art. Such methods include those described by Jaques J, Collet A, & Wilen S in “Enantiomers, Racemates, and Resolutions”, John Wiley and Sons, New York (1981).

Optical active compounds can also be prepared from optical active starting materials or intermediates.

### Labelled Compounds

The compounds of the invention may be used in their labelled or unlabelled form. In the context of this invention the labelled compound has one or more atoms replaced by an atom having an atomic mass or mass number different from the atomic mass or mass number usually found in nature. The labelling will allow easy quantitative detection of said compound.

The labelled compounds of the invention may be useful as diagnostic tools, radio tracers, or monitoring agents in various diagnostic methods, and for in vivo receptor imaging.

The labelled isomer of the invention preferably contains at least one radionuclide as a label. Positron emitting radionuclides are all candidates for usage. In the context of this invention the radionuclide is preferably selected from ²H (deuterium), ³H (tritium), ¹¹C, ¹³C, ¹⁴C, ¹³¹I, ¹²⁵I, ¹²³I, and ¹⁸F.

The physical method for detecting the labelled isomer of the present invention may be selected from Position Emission Tomography (PET), Single Photon Imaging Computed Tomography (SPECT), Magnetic Resonance Spectroscopy (MRS), Magnetic Resonance Imaging (MRI), and Computed Axial X-ray Tomography (CAT), or combinations thereof.

### Methods of Preparation

The chemical compounds of the invention may be prepared by conventional methods for chemical synthesis, e.g. those described in the working examples. The starting materials for the processes described in the present application are known or may readily be prepared by conventional methods from commercially available chemicals.

Also one compound of the invention can be converted to another compound of the invention using conventional methods.

The end products of the reactions described herein may be isolated by conventional techniques, e.g. by extraction, crystallisation, distillation, chromatography, etc.

### Biological Activity

Compounds of the invention may be tested for their ability to inhibit reuptake of the monoamines dopamine, noradrenaline and serotonin in synaptosomes e.g. such as described in WO 97/30997 (NeuroSearch A/S). Based on the balanced activity observed in these tests the compound of the invention is considered useful for the treatment, prevention or alleviation of a disease or a disorder or a condition of a mammal, including a human, which disease, disorder or condition is responsive to inhibition of monoamine neurotransmitter re-uptake in the central nervous system.

In a special embodiment, the compounds of the invention are considered useful for the treatment, prevention or alleviation of: mood disorder, depression, atypical depression, depression secondary to pain, major depressive disorder, dysthymic disorder, bipolar disorder, bipolar I disorder, bipolar II disorder, cyclothymic disorder, mood disorder due to a general medical condition, substance-induced mood disorder, pseudodementia, Ganser's syndrome, obsessive compulsive disorder, panic disorder, panic disorder without agoraphobia, panic disorder with agoraphobia, agoraphobia without history of panic disorder, panic attack, memory deficits, memory loss, attention deficit hyperactivity disorder, obesity, anxiety, generalized anxiety disorder, eating disorder, Parkinson's disease, parkinsonism, dementia, dementia of ageing, senile dementia, Alzheimer's disease, Down's syndrome, acquired immunodeficiency syndrome dementia complex, memory dysfunction in ageing, specific phobia, social phobia, social anxiety disorder, post-traumatic stress disorder, acute stress disorder, drug addiction, drug abuse, drug abuse liability, cocaine abuse, nicotine abuse, tobacco abuse, alcohol addiction, alcoholism, kleptomania, withdrawal symptoms caused by termination of use of addictive substances, pain, chronic pain, inflammatory pain, neuropathic pain, migraine pain, tension-type headache, chronic tension-type headache, pain associated with depression, fibromyalgia, arthritis, osteoarthritis, rheumatoid arthritis, back pain, cancer pain, irritable bowel pain, irritable bowel syndrome, post-operative pain, post-mastectomy pain syndrome (PMPS), post-stroke pain, drug-induced neuropathy, diabetic neuropathy, sympathetically-maintained pain, trigeminal neuralgia, dental pain, myofacial pain, phantom-limb pain, bulimia, premen-strual syndrome, premenstrual dysphoric disorder, late luteal phase syndrome, post-traumatic syndrome, chronic fatigue syndrome, persistent vegetative state, urinary incontinence, stress incontinence, urge incontinence, nocturnal incontinence, sexual dysfunction, premature ejaculation, erectile difficulty, erectile dysfunction, premature female orgasm, restless leg syndrome, periodic limb movement disorder, eating disorders, anorexia nervosa, sleep disorders, pervasive developmental disorders, autism, Asperger's disorder, Rett's disorder, childhood disintegrative disorder, learning disabilities, motor skills disorders, mutism, trichotillomania, narcolepsy, post-stroke depression, stroke-induced brain damage, stroke-induced neuronal damage, Gilles de la Tourettes disease, tinnitus, tic disorders, body dysmorphic disorders, oppositional defiant disorder or post-stroke disabilities. In a preferred embodiment, the compounds are considered useful for the treatment, prevention or alleviation of depression.

It is at present contemplated that a suitable dosage of the active pharmaceutical ingredient (API) is within the range of from about 0.1 to about 1000 mg API per day, e.g. from about 10 to about 500 mg API per day, e.g. of from about 30 to about 100 mg

API per day, dependent, however, upon the exact mode of administration, the form in which it is administered, the indication considered, the subject and in particular the body weight of the subject involved, and further the preference and experience of the physician or veterinarian in charge.

Compounds of the invention show a biological activity in the sub-micromolar and micromolar range, i.e. of from below 1 to about 100 μM.

### Pharmaceutical Compositions

In another aspect the invention provides novel pharmaceutical compositions comprising a therapeutically effective amount of the chemical compound of the invention.

While a chemical compound of the invention for use in therapy may be administered in the form of the raw chemical compound, it is preferred to introduce the active ingredient, optionally in the form of a physiologically acceptable salt, in a pharmaceutical composition together with one or more adjuvants, excipients, carriers, buffers, diluents, and/or other customary pharmaceutical auxiliaries.

In one embodiment, the invention provides pharmaceutical compositions comprising the chemical compound of the invention, or a pharmaceutically acceptable salt or derivative thereof, together with one or more pharmaceutically acceptable carriers, and, optionally, other therapeutic and/or prophylactic ingredients, known and used in the art. The carrier(s) must be “acceptable” in the sense of being compatible with the other ingredients of the formulation and not harmful to the recipient thereof.

The pharmaceutical composition of the invention may be administered by any convenient route, which suits the desired therapy. Examples of routes of administration include oral administration, in particular in tablet, in capsule, in dragé, in powder, or in liquid form, and parenteral administration, in particular cutaneous, subcutaneous, intramuscular, or intravenous injection. The pharmaceutical composition of the invention can be manufactured by any skilled person by use of standard methods and conventional techniques appropriate to the desired formulation. When desired, compositions adapted to give sustained release of the active ingredient may be employed.

Further details on techniques for formulation and administration may be found in the latest edition of Remington's Pharmaceutical Sciences (Maack Publishing Co., Easton, Pa.).

The actual dosage depends on the nature and severity of the disease being treated, and is within the discretion of the physician, and may be varied by titration of the dosage to the particular circumstances of this invention to produce the desired therapeutic effect. However, it is presently contemplated that pharmaceutical compositions containing of from about 0.1 to about 500 mg of active ingredient per individual dose, preferably of from about 1 to about 100 mg, most preferred of from about 1 to about 10 mg, are suitable for therapeutic treatments.

The active ingredient may be administered in one or several doses per day. A satisfactory result can, in certain instances, be obtained at a dosage as low as 0.1 μg/kg i.v. and 1 μg/kg p.o. The upper limit of the dosage range is presently considered to be about 10 mg/kg i.v. and 100 mg/kg p.o. Preferred ranges are from about 0.1 μg/kg to about 10 mg/kg/day i.v., and from about 1 μg/kg to about 100 mg/kg/day p.o.

### Methods of Therapy

In another aspect the invention provides a method for the treatment, prevention or alleviation of a disease or a disorder or a condition of a living animal body, including a human, which disease, disorder or condition is responsive to inhibition of monoamine neurotransmitter re-uptake in the central nervous system, and which method comprises administering to such a living animal body, including a human, in need thereof an effective amount of a chemical compound of the invention.

It is at present contemplated that suitable dosage ranges are 0.1 to 1000 milligrams daily, 10-500 milligrams daily, and e.g. 30-100 milligrams daily, dependent as usual upon the exact mode of administration, form in which administered, the indication toward which the administration is directed, the subject involved and the body weight of the subject involved, and further the preference and experience of the physician or veterinarian in charge.

### EXAMPLES

The invention is further illustrated with reference to the following examples, which are not intended to be in any way limiting to the scope of the invention as claimed.

### Preparatory Examples

### Example 1

### 2-Benzyloxy-6-bromo-naphthalene (1a)

6-Bromonaphtol (25.2 g, 113 mmol) and benzyl chloride (39.0 mL, 339 mmol) were dissolved in N,N-dimethylformamide (125 mL). Potassium carbonate (46.9 g, 399 mmol) and sodium iodide (1.69 g, 11.3 mmol) were added and the mixture was stirred at 80° C. overnight. The reaction mixture was concentrated in vacuo. Water and ethyl acetate were added and the phases were separated. The aqueous phase was extracted with ethyl acetate. The combined organic phases were washed with brine, dried over sodium sulphate filtrated and concentrated in vacuo. Recrystallisation from ethanol gave 2-benzyloxy-6-bromo-naphthalene (27.8 g, 78%) as a white crystalline compound.

### Example 2

### 4-[(3-Chloro-phenylamino)-methyl]-piperidine-1-carboxylic acid tert-butyl ester (2a)

4-Aminomethyl-piperidine-1-carboxylic acid tert-butyl ester (3.0 g, 14.0 mmol), 3-bromochlorobenzene (2.0 mL, 16.8 mmol) and sodium tert-butoxide (2.06 g, 21.0 mmol) were dissolved in toluene (150 mL) and argon was bubbled through. 4,5-Bis(diphenylphosphino)-9,9-dimethylxanthene (Xanthphos) (251 mg, 0.42 mmol) and tris(dibenzylidineacetone)dipalladium (Pd₂(dba)₃) (128 mg, 0.14 mmol) were added and the mixture was stirred at 110° C. for 3 hours. Aqueous sodium hydrogencarbonate was added and the mixture was filtrated. The organic layer was washed with brine, dried over sodium sulphate in combination with activated charcoal. The mixture was filtrated through a pad of celite and concentrated in vacuo to give the crude product as an orange sticky oil. 4-[(3-Chloro-phenylamino)-methyl]-piperidine-1-carboxylic acid tert-butyl ester (2.95 g, 65%) was crystallised from ethyl acetate/heptane to give a white solid.

Compounds 2b-2f were prepared in parallel with compound 2a from 4-amino-methyl-piperidine-1-carboxylic acid tert-butyl ester and the various aryl halides as shown in the table below.

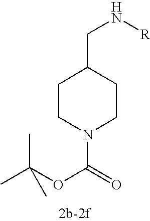

| Compound | | | |
|---|---|---|---|
| No. | R | Aryl Halide | Yield (%) |
| 2b | Phenyl | bromobenzene | 57 |
| 2c | 3,4-dichlorophenyl | 1-bromo-3,4- | 70 |
| | | dichlorobenzene | |
| 2d | 5-chloro-pyridin-2-yl | 2-bromo-5- | 23 |
| | | chloropyridine | |
| 2e | 6-methoxy-naphthalen- | 2-bromo-6- | 40 |
| | 2-yl | methoxynaphthalene | |
| 2f | 6-benzyloxy- | 2-benzyloxy-6-bromo- | 57 |
| | naphthalen-2-yl | bromo-naphthalene | |

### Example 3

### 4-(Pyridin-2-ylaminomethyl)-piperidine-1-carboxylic acid tert-butyl ester (3a)

4-Aminomethyl-piperidine-1-carboxylic acid tert-butyl ester (2.69 g, 12.53 mmol) and pyridine (0.61 mL, 7.52 mmol) were mixed. 2-Bromopyridine (0.60 mL, 6.27 mmol) was added and the reaction mixture was stirred at 125° C. overnight. Dichloromethane and sodium hydrogencarbonate were added and the phases separated. The aqueous phase was extracted with dichloromethane. The combined organic phases were dried over sodium sulphate, filtrated and concentrated in vacuo. The crude product was purified by flash chromatography (ethyl acetate/heptane as eluent) to give 4-(pyridin-2-ylaminomethyl)-piperidine-1-carboxylic acid tert-butyl ester (0.82 g, 45%) as a white solid.

### Example 4

### 4-{[(3-Chloro-phenyl)-propionyl-amino]-methyl}-piperidine-1-carboxylic acid tert-butyl ester (4a)

4-[(3-Chloro-phenylamino)-methyl]-piperidine-1-carboxylic acid tert-butyl ester (2.92 g, 9.0 mmol) and triethylamine (1.91 mL, 13.5 mmol) were dissolved in dichloromethane (80 mL) and cooled to 0° C. Propionyl chloride (0.88 mL, 9.89 mmol) was added dropwise and the reaction mixture was stirred at room temperature for 2 hours. Water was added and the organic layer was washed with aqueous sodium hydrogencarbonate, water and brine. The organic layer was dried over sodium sulphate, filtrated and concentrated in vacuo to give 4-{[(3-chloro-phenyl)-propionyl-amino]-methyl}-piperidine-1-carboxylic acid tert-butyl ester (3.0 g, 88%) as a yellow oil.

Compounds 4b-4g were prepared in parallel with compound 4a as shown in the table below.

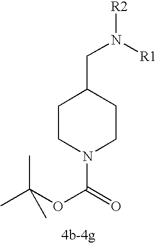

| Com- | | | | |
|---|---|---|---|---|
| pound | | | Starting | Yield |
| No. | R1 | R2 | material | % |
| 4b | phenyl | propionyl | 2b | 100 |
| 4c | 3,4-dichlorophenyl | propionyl | 2c | 64 |
| 4d | 2-pyridyl | propionyl | 3a | 44 |
| 4e | 5-chloro-pyridin-2-yl | propionyl | 2d | 49 |
| 4f | 6-methoxy- | propionyl | 2e | 92 |
| | naphthalen-2-yl | | | |
| 4g | 6-benzyloxy- | propionyl | 2f | 96 |
| | naphthalen-2-yl | | | |
| 4h | 3,4-dichlorophenyl | acetyl | 2c | 100 |
| 4i | 3,4-dichlorophenyl | methoxyacetyl | 2c | 87 |

### Example 5

### N-(3-Chloro-phenyl)-N-piperidin-4-ylmethyl-propionamide (5a)

4-{[(3-Chloro-phenyl)-propionyl-amino]-methyl}-piperidine-1-carboxylic acid tert-butyl ester (4a) (3.0 g, 7.88 mmol) was dissolved in dichloromethane (80 mL) and cooled to 0° C. Trifluoroacetic acid (8.0 mL, 107 mmol) in dichlormethane (20 mL) was added drop wise and the mixture was stirred for one hour at room temperature. The reaction mixture was concentrated and triturated with toluene, ethyl acetate and diethylether. The resulting solid was filtrated and washed with diethyl ether to give N-(3-chloro-phenyl)-N-piperidin-4-ylmethyl-propionamide (3.1 g, 100%) as the trifluoro-acetic acid salt Mp.=94-96° C.

LC-ESI-HRMS of [M+H]+ shows 281.1431 Da. Calc. 281.142066 Da, dev. 3.7 ppm

Compounds 5b-5g were prepared in parallel with compound 5a as shown in the table below.

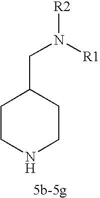

| Com- | | | | |
|---|---|---|---|---|
| pound | | | Starting | Yield |
| No. | R1 | R2 | material | (%) |
| 5b | phenyl | propionyl | 4b | 92 |
| 5c | 3,4-dichlorophenyl | propionyl | 4c | 80 |
| 5d | 2-pyridyl | propionyl | 4d | 66 |
| 5e | 5-chloro-pyridin-2-yl | propionyl | 4e | 79 |
| 5f | 6-methoxy-naphthalen- | propionyl | 4f | 78 |
| | 2-yl | | | |
| 5g | 6-benzyloxy-naphthalen- | propionyl | 4g | 85 |
| | 2-yl | | | |
| 5h | 3,4-dichlorophenyl | acetyl | 4h | 75 |
| 5i | 3,4-dichlorophenyl | methoxyacetyl | 4i | 90 |

### N-Phenyl-N-piperidin-4-ylmethyl-propionamide trifluoroacetic acid salt (5b)

Mp.=98-100° C.

LC-ESI-HRMS of [M+H]+ shows 247.1802 Da. Calc. 247.181038 Da, dev. −3.4 ppm

### N-Piperidin-4-ylmethyl-N-pyridin-2-yl-propionamide trifluoroacetic acid salt (5d)

Mp.=108-110° C.

LC-ESI-HRMS of [M+H]+ shows 248.1762 Da. Calc. 248.176287 Da, dev. −0.4

### N-(3,4-Dichloro-phenyl)-N-piperidin-4-ylmethyl-propionamide trifluoroacetic acid salt (5c)

Mp.=123-125° C.

LC-ESI-HRMS of [M+H]+ shows 315.1015 Da. Calc. 315.103094 Da, dev. −5.1 ppm

### N-(6-Methoxy-naphthalen-2-yl)-N-piperidin-4-ylmethyl-propionamide trifluoro-acetic acid salt (5f)

Mp.=168-170° C.

LC-ESI-HRMS of [M+H]+ shows 327.2077 Da. Calc. 327.207253 Da, dev. 1.4 ppm

### N-(5-Chloro-pyridin-2-yl)-N-piperidin-4-ylmethyl-propionamide trifluoroacetic acid salt (5e)

Mp.=134-136° C.

LC-ESI-HRMS of [M+H]+ shows 282.137 Da. Calc. 282.137315 Da, dev. −1.1 ppm

### N-(6-Benzyloxy-naphthalen-2-yl)-N-piperidin-4-ylmethyl-propionamide trifluoroacetic acid salt (5g)

Mp.=151-153° C.

LC-ESI-HRMS of [M+H]+ shows 403.2369 Da. Calc. 403.238553 Da, dev. −4.1 ppm

### N-(3,4-Dichloro-phenyl)-N-piperidin-4-ylmethyl-acetamide trifluoroacetic acid salt (5h)

Mp.=172-174° C.

LC-ESI-HRMS of [M+H]+ shows 301.0887 Da. Calc. 301.087444 Da, dev. 4.2 ppm

### N-(3,4-Dichloro-phenyl)-2-methoxy-N-piperidin-4-ylmethyl-acetamide trifluoro-acetic acid salt (51)

Mp.=115-117° C.

LC-ESI-HRMS of [M+H]+ shows 331.0987 Da. Calc. 331.098009 Da, dev. 2.1 ppm

### Example 6

### N-(6-Hydroxy-naphthalen-2-yl)-N-piperidin-4-ylmethyl-propionamide trifluoro-acetic acid salt (6a)

N-(6-Benzyloxy-naphthalen-2-yl)-N-piperidin-4-ylmethyl-propionamide (312 mg, 0.60 mmol) was dissolved in methanol (5 mL), palladium on carbon was added and the reaction mixture was stirred under a hydrogen atmosphere for 3 hours.

The reaction mixture was filtrated and concentrated in vacuo to give N-(6-hydroxy-naphthalen-2-yl)-N-piperidin-4-ylmethyl-propionamide (177 mg, 69%) as a white solid as the trifluoroacetic acid salt.

Mp.=101-103° C.

LC-ESI-HRMS of [M+H]+ shows 313.1932 Da. Calc. 313.191603 Da, dev. 5.1 ppm

### Example 7

### N-(3,4-Dichloro-phenyl)-N-(1-methyl-piperidin-4-ylmethyl)-propionamide fumaric acid salt (7a)

N-(3,4-Dichloro-phenyl)-N-piperidin-4-ylmethyl-propionamide (500 mg, 1.16 mmol) was dissolved in dichloroethane (5 mL) paraformaldehyde (0.13 mL, 1.51 mmol) was added and the mixture was stirred for 1 hour at room temperature. Sodium triacetoxyborohydride (509 mg, 2.33 mmol) was added portion wise and the reaction mixture was stirred over night. Aqueous sodium hydrogen carbonate was added followed by ex-traction with dichloromethane. The combined organic phases were dried over sodium sulphate filtrated and concentrated in vacuo. The orange oil was dissolved in dichloro-methane/methanol and fumaric acid was added. The mixture was concentrated in vacuo and co-evaporated with diethylether. The white solid was suspended in a mixture of ethyl acetate and heptane and collected by filtration to give N-(3,4-dichloro-phenyl)-N-(1-methyl-piperidin-4-ylmethyl)-propionamide fumaric acid salt (343 mg, 66%)

LC-ESI-HRMS of [M+H]+ shows 329.1172 Da. Calc. 329.118744 Da, dev. −4.7 ppm

### Test Example

### In Vitro Inhibition Activity

A number of compounds were tested for their ability to inhibit the reuptake of the monoamine neurotransmitters dopamine (DA), noradrenaline (NA) and serotonine (5-HT) in synaptosomes as described in WO 97/16451.

The test values are given as IC₅₀ (the concentration (μM) of the test substance which inhibits the specific binding of ³H-DA, ³H-NA, or ³H-5-HT by 50%).

Test results obtained by testing selected compounds of the present invention appear from the below table:

TABLE 1

| | 5-HT-uptake | DA-uptake | NA-uptake |
|---|---|---|---|
| Test compound | IC₅₀(μM) | IC₅₀(μM) | IC₅₀(μM) |
| N-(3,4-Dichloro-phenyl)- | 0.0066 | 0.086 | 0.0072 |
| N-piperidin-4-ylmethyl- | | | |
| propionamide (5c) | | | |
| N-(6-Hydroxy-naphthalen-2- | 0.047 | 0.49 | 0.0081 |
| yl)-N-piperidin-4-ylmethyl- | | | |
| propionamide (6a) | | | |

1. A compound of Formula I: 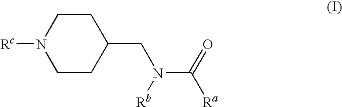
any of its stereoisomers or any mixture of its stereoisomers, or a pharmaceutically acceptable salt thereof; wherein
R^{a} represents hydrogen or alkyl; which alkyl is optionally substituted with one or more substituents independently selected from the group consisting of halo, trifluoromethyl, trifluoromethoxy, cyano, hydroxy, amino, nitro, alkoxy, cycloalkoxy, alkyl, cycloalkyl, cycloalkylalkyl, alkenyl and alkynyl;
R^{b} represents an aryl or heteroaryl group; which aryl or heteroaryl group is optionally substituted with one or more substituents independently selected from the group consisting of halo, trifluoromethyl, trifluoromethoxy, cyano, nitro, hydroxy, alkoxy, cycloalkoxy, phenyloxy, benzyloxy, alkoxyalkyl, cycloalkoxyalkyl, methylenedioxy, ethylenedioxy, alkyl, cycloalkyl, cycloalkylalkyl, alkenyl, alkynyl, sulfanyl, thioalkoxy, —NR′R″, —(C═O)NR′R″ or —NR′(C═O)R″; wherein R′ and R″ independent of each other are hydrogen or alkyl;
R^{c} represents hydrogen or alkyl;
with the proviso that the compound is not
N-(4-Chloro-phenyl)-N-piperidin-4-ylmethyl-propionamide;
N-Benzyl-N-piperidin-4-ylmethyl-propionamide;
N-Phenyl-N-piperidin-4-ylmethyl-propionamide;
N-(3-Methoxy-phenyl)-N-piperidin-4-ylmethyl-propionamide;
N-Benzo[1,3]dioxol-5-yl-N-piperidin-4-ylmethyl-propionamide;
N-(4-Methoxy-phenyl)-N-piperidin-4-ylmethyl-propionamide;
N-(4-Fluoro-phenyl)-N-piperidin-4-ylmethyl-propionamide; or
N-Phenyl-N-piperidin-4-ylmethyl-acetamide.

2. The compound according to claim 1, any of its stereoisomers or any mixture of its stereoisomers, or a pharmaceutically acceptable salt thereof, wherein R^{a} represents alkyl.

3. The compound according to claim 1 or 2, any of its stereoisomers or any mixture of its stereoisomers, or a pharmaceutically acceptable salt thereof, wherein R^{b} represents optionally substituted phenyl.

4. The compound according to claim 1 or 2, any of its stereoisomers or any mixture of its stereoisomers, or a pharmaceutically acceptable salt thereof, wherein R^{b} represents optionally substituted naphthyl.

5. The compound according to claim 1 or 2, any of its stereoisomers or any mixture of its stereoisomers, or a pharmaceutically acceptable salt thereof, wherein R^{b} represents optionally substituted pyridyl.

6. The compound according to claim 1, any of its stereoisomers or any mixture of its stereoisomers, or a pharmaceutically acceptable salt thereof, wherein R^{c} represents hydrogen.

7. A compound according to claim 1 which is
N-(3-Chloro-phenyl)-N-piperidin-4-ylmethyl-propionamide;
N-Phenyl-N-piperidin-4-ylmethyl-propionamide;
N-Piperidin-4-ylmethyl-N-pyridin-2-yl-propionamide;
N-(3,4-Dichloro-phenyl)-N-piperidin-4-ylmethyl-propionamide;
N-(6-Methoxy-naphthalen-2-yl)-N-piperidin-4-ylmethyl-propionamide;
N-(5-Chloro-pyridin-2-yl)-N-piperidin-4-ylmethyl-propionamide;
N-(6-Benzyloxy-naphthalen-2-yl)-N-piperidin-4-ylmethyl-propionamide;
N-(6-Hydroxy-naphthalen-2-yl)-N-piperidin-4-ylmethyl-propionamide;
any of its stereoisomers or any mixture of its stereoisomers, or
a pharmaceutically acceptable salt thereof.

8. A compound according to claim 1 which is
N-(3,4-Dichloro-phenyl)-N-piperidin-4-ylmethyl-acetamide;
N-(3,4-Dichloro-phenyl)-2-methoxy-N-piperidin-4-ylmethyl-acetamide;
N-(3,4-Dichloro-phenyl)-N-(1-methyl-piperidin-4-ylmethyl)-propionamide;
any of its stereoisomers or any mixture of its stereoisomers, or
a pharmaceutically acceptable salt thereof.

9. A pharmaceutical composition, comprising a therapeutically effective amount of a compound according to any claim 1, or the compound
N-(4-Chloro-phenyl)-N-piperidin-4-ylmethyl-propionamide;
N-Benzyl-N-piperidin-4-ylmethyl-propionamide;
N-Phenyl-N-piperidin-4-ylmethyl-propionamide;
N-(3-Methoxy-phenyl)-N-piperidin-4-ylmethyl-propionamide;
N-Benzo[1,3]dioxol-5-yl-N-piperidin-4-ylmethyl-propionamide;
N-(4-Methoxy-phenyl)-N-piperidin-4-ylmethyl-propionamide;
N-(4-Fluoro-phenyl)-N-piperidin-4-ylmethyl-propionamide;
N-Phenyl-N-piperidin-4-ylmethyl-acetamide, or
any of its stereoisomers or any mixture of its stereoisomers, or a pharmaceutically acceptable salt thereof, together with at least one pharmaceutically acceptable carrier, excipient or diluent.

10. (canceled)

11. (canceled)

12. (canceled)

13. A method for treatment, prevention or alleviation of a disease or a disorder or a condition of a living animal body, including a human, which disorder, disease or condition is responsive to inhibition of monoamine neurotransmitter re-uptake in the central nervous system, which method comprises the step of administering to such a living animal body in need thereof a therapeutically effective amount of a compound according to claim 1, or the compound
N-(4-Chloro-phenyl)-N-piperidin-4-ylmethyl-propionamide;
N-Benzyl-N-piperidin-4-ylmethyl-propionamide;
N-Phenyl-N-piperidin-4-ylmethyl-propionamide;
N-(3-Methoxy-phenyl)-N-piperidin-4-ylmethyl-propionamide;
N-Benzo[1,3]dioxol-5-yl-N-piperidin-4-ylmethyl-propionamide;
N-(4-Methoxy-phenyl)-N-piperidin-4-ylmethyl-propionamide;
N-(4-Fluoro-phenyl)-N-piperidin-4-ylmethyl-propionamide; or
N-Phenyl-N-piperidin-4-ylmethyl-acetamide, or
any of its stereoisomers or any mixture of its stereoisomers, or a pharmaceutically acceptable salt thereof.
